(19) United States
(12) Patent Application Publication
(10) Pub. No.: US 2024/0157130 A1
(43) Pub. Date: May 16, 2024

(54) IMPLANTABLE MEDICAL SYSTEMS FOR CANCER TREATMENT WITH THERMAL MANAGEMENT FEATURES

(71) Applicant: Cardiac Pacemakers, Inc., St. Paul, MN (US)

(72) Inventors: Devon N. Arnholt, Shoreview, MN (US); Michael J. Kane, St. Paul, MN (US); Brian L. Schmidt, White Bear Lake, MN (US)

(73) Assignee: MAYO FOUNDATION FOR MEDICAL EDUCATION AND RESEARCH, Rochester, MN (US)

(21) Appl. No.: 18/386,895

(22) Filed: Nov. 3, 2023

Related U.S. Application Data
(60) Provisional application No. 63/424,372, filed on Nov. 10, 2022.

(51) Int. Cl. A61N 1/36; A61N 1/02; A61N 1/05; A61N 1/20

(52) U.S. Cl. CPC A61N 1/36002; A61N 1/025; A61N 1/05; A61N 1/205

(57) ABSTRACT

Embodiments herein relate to implantable systems for cancer treatment and related methods. In an embodiment, an implantable system for cancer treatment can be included having a therapy output circuit configured to generate an electrical output for one or more electrodes to create one or more electric fields. The implantable system can include control circuitry that causes the therapy output circuit to generate the one or more electric fields at frequencies between 10 kHz and 1 MHz within a bodily tissue. The one or more electric fields can be effective to prevent and./or disrupt cellular mitosis in a cell. The implantable system can further include a therapy zone temperature sensor. The implantable system can be configured to measure the temperature and/or record the temperature data of a patient over time. The temperature data can include tissue temperature and time stamps of the same. Other embodiments are also included herein.

This application claims the benefit of U.S. Provisional Application No. 63/424,372, filed Nov. 10, 2022, the content of which is herein incorporated by reference in its entirety.

### FIELD

Embodiments herein relate to implantable systems for cancer treatment and related methods.

### BACKGROUND

According to the American Cancer Society, cancer accounts for nearly 25% of the deaths that occur in the United States each year. The current standard of care for cancerous tumors can include first-line therapies such as surgery, radiation therapy, and chemotherapy. Additional second-line therapies can include radioactive seeding, cryotherapy, hormone or biologics therapy, ablation, and the like. Combinations of first-line therapies and second-line therapies can also be a benefit to patients if one particular therapy on its own is not effective.

Cancerous tumors can form if one normal cell in any part of the body mutates and then begins to grow and multiply too much and too quickly. Cancerous tumors can be a result of a genetic mutation to the cellular DNA or RNA that arises during cell division, an external stimulus such as ionizing or non-ionizing radiation, exposure to a carcinogen, or a result of a hereditary gene mutation. Regardless of the etiology, many cancerous tumors are the result of unchecked rapid cellular division.

Various cancer therapies may have significant side effects on heathy tissue. Such side effects can vary widely depending on the type of cancer therapy but can manifest as anemia, thrombocytopenia, edema, alopecia, infections, neutropenia, lymphedema, cognitive problems, nausea and vomiting, neuropathy, skin and nail changes, sleep problems, urinary and bladder problems, and the like.

### SUMMARY

Embodiments herein relate to implantable systems for cancer treatment and related methods. In a first aspect, an implantable system for cancer treatment can be included having a therapy output circuit, wherein the therapy output circuit can be configured to generate an electrical output for one or more electrodes to create one or more electric fields, control circuitry, wherein the control circuitry causes the therapy output circuit to generate the one or more electric fields at frequencies selected from a range of between 10 kHz to 1 MHz within a bodily tissue, wherein the one or more electric fields can be effective to prevent and/or disrupt cellular mitosis in a cell, and a therapy zone temperature sensor, wherein the implantable system can be configured to measure temperature and/or record temperature data of a patient over time, and the temperature data can include tissue temperatures and time stamps of the same.

In a second aspect, in addition to one or more of the preceding or following aspects, or in the alternative to some aspects, the control circuitry can be configured to process the temperature data and modulate a therapy parameter based on the temperature data.

In a third aspect, in addition to one or more of the preceding or following aspects, or in the alternative to some aspects, the implantable system can be configured to use activity state data of the one or more electrodes and/or operational history of the one or more electrodes when processing temperature data from the therapy zone temperature sensor.

In a fourth aspect, in addition to one or more of the preceding or following aspects, or in the alternative to some aspects, the therapy parameter can include at least one selected from the group consisting of a field strength, a duty cycle, and a therapy vector.

In a fifth aspect, in addition to one or more of the preceding or following aspects, or in the alternative to some aspects, the control circuitry can be configured to process the temperature data and modulate a therapy parameter based on the temperature d ata as part of a closed loop control system.

In a sixth aspect, in addition to one or more of the preceding or following aspects, or in the alternative to some aspects, the therapy parameter can include a closed loop thermal setpoint value.

In a seventh aspect, in addition to one or more of the preceding or following aspects, or in the alternative to some aspects, the implantable system can be configured to index the temperature data based on one or more of direct or indirect measurement, tissue type, and electrode proximity.

In an eighth aspect, in addition to one or more of the preceding or following aspects, or in the alternative to some aspects, the implantable system can be configured to track the amount of time that tissue can be exposed to temperatures above a threshold level.

In a ninth aspect, in addition to one or more of the preceding or following aspects, or in the alternative to some aspects, the implantable system can be configured to modulate a therapy parameter to limit the amount of time that the tissue can be exposed to temperatures above a threshold level.

In a tenth aspect, in addition to one or more of the preceding or following aspects, or in the alternative to some aspects, the implantable system can be configured to reduce a therapy parameter value to limit the amount of time that the tissue can be exposed to temperatures above a threshold level over a specific time period and then later increase the therapy parameter value.

In an eleventh aspect, in addition to one or more of the preceding or following aspects, or in the alternative to some aspects, the implantable system can be configured to issue an alert if the amount of time that tissue can be exposed to temperatures above a threshold level exceeds a predetermined amount of time.

In a twelfth aspect, in addition to one or more of the preceding or following aspects, or in the alternative to some aspects, the implantable system can be configured to increase an intensity of the therapy at times when a core body temperature or a reference body temperature can be lower than a threshold temperature.

In a thirteenth aspect, in addition to one or more of the preceding or following aspects, or in the alternative to some aspects, the implantable system can be configured to increase an intensity of a therapy parameter at times when the patient can be asleep.

In a fourteenth aspect, in addition to one or more of the preceding or following aspects, or in the alternative to some aspects, the implantable system can be configured to decrease an intensity of a therapy parameter at times when the patient can have an elevated core body temperature or reference body temperature.

In a fifteenth aspect, in addition to one or more of the preceding or following aspects, or in the alternative to some aspects, the implantable system can be configured to decrease an intensity of a therapy parameter at times when the patient can have a fever.

In a sixteenth aspect, in addition to one or more of the preceding or following aspects, or in the alternative to some aspects, the therapy zone temperature sensor can be configured to measure or estimate a core body temperature or a reference body temperature when therapy can be turned off or paused.

In a seventeenth aspect, in addition to one or more of the preceding or following aspects, or in the alternative to some aspects, further can include a second temperature sensor, wherein the second temperature sensor can be configured to measure or estimate a core body temperature or reference body temperature of the patient.

In an eighteenth aspect, in addition to one or more of the preceding or following aspects, or in the alternative to some aspects, further can include a third temperature sensor, wherein the third temperature sensor can be configured to measure or estimate a core body temperature or reference body temperature of the patient.

In a nineteenth aspect, in addition to one or more of the preceding or following aspects, or in the alternative to some aspects, the implantable system can be configured to measure and/or receive a core body temperature or a reference body temperature of the patient and increase an intensity of a therapy parameter at times when the core body temperature or reference body temperature can be lower than a threshold temperature.

In a twentieth aspect, in addition to one or more of the preceding or following aspects, or in the alternative to some aspects, further can include a stimulation lead, wherein the therapy zone temperature sensor can be configured to measure a temperature at a site along the stimulation lead.

In a twenty-first aspect, in addition to one or more of the preceding or following aspects, or in the alternative to some aspects, the implantable system can further include a stimulation lead, the stimulation lead can include an electrode, and wherein the therapy zone temperature sensor can be configured to measure a temperature at a site offset from a surface of the electrode.

In a twenty-second aspect, in addition to one or more of the preceding or following aspects, or in the alternative to some aspects, the implantable system compensates for the offset in measuring or estimating tissue temperatures.

In a twenty-third aspect, in addition to one or more of the preceding or following aspects, or in the alternative to some aspects, the therapy zone temperature sensor can be an optical thermal sensor.

In a twenty-fourth aspect, in addition to one or more of the preceding or following aspects, or in the alternative to some aspects, further can include a stimulation lead, wherein the therapy zone temperature sensor can be connected to the stimulation lead.

In a twenty-fifth aspect, in addition to one or more of the preceding or following aspects, or in the alternative to some aspects, further can include stimulation electrodes, wherein the stimulation electrodes can be disposed on the stimulation lead, and wherein the stimulation electrodes can be in electrical communication with the therapy output circuit.

In a twenty-sixth aspect, in addition to one or more of the preceding or following aspects, or in the alternative to some aspects, further can include an accelerometer, wherein the implantable system can be configured to estimate physical activity based on a signal from the accelerometer and modulate a therapy parameter based on the same.

In a twenty-seventh aspect, in addition to one or more of the preceding or following aspects, or in the alternative to some aspects, the implantable system can be configured to modulate the therapy parameter based on estimated physical activity in advance of measured temperature changes.

In a twenty-eighth aspect, in addition to one or more of the preceding or following aspects, or in the alternative to some aspects, the implantable system can be configured to regulate therapy zone temperature limits to contain maximal therapy dose.

In a twenty-ninth aspect, a method of providing electrical stimulation cancer therapy can be included. The method can include generating an electrical output for one or more electrodes to create one or more electric fields, measuring temperature and/or recording temperature data of a patient over time, and processing the temperature data and modulating a therapy parameter based on the temperature data.

In a thirtieth aspect, in addition to one or more of the preceding or following aspects, or in the alternative to some aspects, the method can further include using activity state data of the one or more electrodes and/or operational history of the one or more electrodes when processing the temperature data.

In a thirty-first aspect, in addition to one or more of the preceding or following aspects, or in the alternative to some aspects, the method can further include processing the temperature data and modulate the therapy parameter based on the temperature data as part of a closed loop control system.

In a thirty-second aspect, in addition to one or more of the preceding or following aspects, or in the alternative to some aspects, the method can further include indexing the temperature data based on one or more of direct or indirect measurement, tissue type, and electrode proximity.

In a thirty-third aspect, in addition to one or more of the preceding or following aspects, or in the alternative to some aspects, the method can further include tracking the amount of time that tissue can be exposed to temperatures above a threshold level.

In a thirty-fourth aspect, in addition to one or more of the preceding or following aspects, or in the alternative to some aspects, the method can further include modulating a therapy parameter to limit the amount of time that the tissue can be exposed to temperatures above a threshold level.

In a thirty-fifth aspect, in addition to one or more of the preceding or following aspects, or in the alternative to some aspects, the method can further include reducing a therapy parameter value to limit the amount of time that the tissue can be exposed to temperatures above a threshold level over a specific time period and then later increasing the therapy parameter value.

In a thirty-sixth aspect, in addition to one or more of the preceding or following aspects, or in the alternative to some aspects, the method can further include issuing an alert if the amount of time that tissue can be exposed to temperatures above a threshold level exceeds a predetermined amount of time.

In a thirty-seventh aspect, in addition to one or more of the preceding or following aspects, or in the alternative to some aspects, the method can further include increasing an intensity of the therapy at times when a core body temperature or a reference body temperature can be lower than a threshold temperature.

In a thirty-eighth aspect, in addition to one or more of the preceding or following aspects, or in the alternative to some aspects, the method can further include increasing an intensity of the therapy parameter at times when the patient can be asleep.

In a thirty-ninth aspect, in addition to one or more of the preceding or following aspects, or in the alternative to some aspects, the method can further include decreasing an intensity of the therapy parameter at times when the patient can have an elevated core body temperature or reference body temperature.

In a fortieth aspect, in addition to one or more of the preceding or following aspects, or in the alternative to some aspects, the method can further include decreasing an intensity of the therapy parameter at times when the patient can have a fever.

In a forty-first aspect, in addition to one or more of the preceding or following aspects, or in the alternative to some aspects, the method can further include measuring or estimate a core body temperature or a reference body temperature when therapy can be turned off or paused.

In a forty-second aspect, in addition to one or more of the preceding or following aspects, or in the alternative to some aspects, the method can further include measuring or estimate a core body temperature or reference body temperature of the patient.

In a forty-third aspect, in addition to one or more of the preceding or following aspects, or in the alternative to some aspects, further can include: measuring and/or receive a core body temperature or a reference body temperature of the patient and increasing an intensity of the therapy parameter at times when the core body temperature or reference body temperature can be lower than a threshold temperature.

In a forty-fourth aspect, in addition to one or more of the preceding or following aspects, or in the alternative to some aspects, the method can further include measuring a temperature at a site along a stimulation lead.

In a forty-fifth aspect, in addition to one or more of the preceding or following aspects, or in the alternative to some aspects, the method can further include measuring a temperature at a site offset from a surface of an electrode.

In a forty-sixth aspect, in addition to one or more of the preceding or following aspects, or in the alternative to some aspects, the method can further include measuring a temperature at a site underneath a surface of an electrode.

In a forty-seventh aspect, in addition to one or more of the preceding or following aspects, or in the alternative to some aspects, the method can further include estimating physical activity based on a signal from an accelerometer and modulating a therapy parameter based on the same.

In a forty-eighth aspect, in addition to one or more of the preceding or following aspects, or in the alternative to some aspects, estimating physical activity based on a signal from an accelerometer and modulating a therapy parameter based on the same includes modulating the therapy parameter based on estimated physical activity in advance of measured temperature changes.

In a forty-ninth aspect, in addition to one or more of the preceding or following aspects, or in the alternative to some aspects, the method can further include regulating therapy zone temperature limits to contain maximal therapy dose.

In a fiftieth aspect, an implantable system for cancer treatment can be included having a housing, a therapy output circuit, and control circuitry. The therapy output circuit can be configured to generate one or more electric fields. The control circuitry can cause the therapy output circuit to generate the one or more electric fields at frequencies selected from a range of between 10 kHz to 1 MHz within a bodily tissue. The one or more electric fields can be effective to prevent and/or disrupt cellular mitosis in a cell. The control circuitry and the therapy output circuit can be disposed within the housing. A first temperature sensor can be configured to measure a temperature of tissue at a site of therapy. A stimulation lead with stimulation electrodes can be included wherein the stimulation electrodes can be disposed on the stimulation lead. The stimulation electrodes can be in electrical communication with the therapy output circuit. A second temperature sensor can be included and can be configured to measure a core temperature or reference temperature of a patient.

In a fifty-first aspect, in addition to one or more of the preceding or following aspects, or in the alternative to some aspects, the control circuitry can be configured to modulate a therapy parameter based on the core temperature or reference temperature.

In a fifty-second aspect, in addition to one or more of the preceding or following aspects, or in the alternative to some aspects, the therapy parameter can include at least one selected from the group consisting of a field strength, a duty cycle, and a therapy vector.

In a fifty-third aspect, in addition to one or more of the preceding or following aspects, or in the alternative to some aspects, the therapy parameter can include a closed loop thermal setpoint value.

In a fifty-fourth aspect, in addition to one or more of the preceding or following aspects, or in the alternative to some aspects, the second temperature sensor can be disposed remotely from the housing and the stimulation lead.

In a fifty-fifth aspect, in addition to one or more of the preceding or following aspects, or in the alternative to some aspects, the second temperature sensor can be in wireless communication with the control circuitry.

In a fifty-sixth aspect, in addition to one or more of the preceding or following aspects, or in the alternative to some aspects, the implantable system can be configured to measure temperature and/or record temperature data of a patient over time.

In a fifty-seventh aspect, in addition to one or more of the preceding or following aspects, or in the alternative to some aspects, the temperature data can include tissue temperatures and time stamps of the same.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following drawings, in which:

FIG. 1 is a schematic view of a medical device implanted in a patient in accordance with various embodiments herein.

FIG. 2 is a schematic view of a medical device implanted in a patient in accordance with various embodiments herein.

FIG. 3 is a schematic view of a placement of various cancer therapy stimulation leads in a region of a tumor resection site in accordance with various embodiments herein.

FIG. 4 is a schematic view of a placement of various cancer therapy stimulation leads in a region of a tumor resection site in accordance with various embodiments herein.

FIG. 5 is a schematic view of a placement of various cancer therapy stimulation leads and a temperature sensor in a region of a tumor resection site in accordance with various embodiments herein.

FIG. 6 is a top view of a placement of various cancer therapy stimulation leads in a region of a tumor resection site in accordance with various embodiments herein.

FIG. 7 is a top view of a placement of three cancer therapy stimulation leads in a region of a tumor resection site in accordance with various embodiments herein.

FIG. 8 is a flow chart depicting a method in accordance with various embodiments herein.

FIG. 9 is a flow chart depicting a method in accordance with various embodiments herein.

FIG. 10 is a schematic view of a cancer therapy stimulation lead in accordance with various embodiments herein.

FIG. 11 is a cross-sectional schematic view of a cancer therapy stimulation lead in accordance with various embodiments herein.

FIG. 12 is a schematic cross-sectional view of a medical device in accordance with various embodiments herein.

FIG. 13 is a schematic diagram of components of a medical device in accordance with various embodiments herein.

While embodiments are susceptible to various modifications and alternative forms, specifics thereof have been shown by way of example and drawings, and will be described in detail. It should be understood, however, that the scope herein is not limited to the particular embodiments described. On the contrary, the intention is to cover modifications, equivalents, and alternatives falling within the spirit and scope herein.

### DETAILED DESCRIPTION

As described above, various cancer therapies may have side effects on healthy tissue. Such side effects can be significant and thus it is advantageous to minimize them as much as possible.

Cancer therapies including electrical stimulation may generate an amount of waste heat. For example, electrical stimulation to prevent and/or disrupt cellular mitosis may generate heat. While the amount of heat generated for electrical stimulation to prevent and/or disrupt cellular mitosis may be substantially less than with other techniques such as electrical stimulation for tissue ablation, the heat may be enough to raise the temperature of tissues to levels resulting in negative tissue effects and/or tissue damage. Issues with heat exposure can be particularly critical in the context of treating tissue inside the head such as in the case of a brain tumor and/or the treatment of a tumor resection site.

Embodiments of systems herein can be configured to measure temperature and/or record temperature data of patient tissue over time to track the amount of heat exposure of such tissue. Temperature data can include tissue temperatures and times of exposure of the same. Such tracking can provide various benefits. For example, a clinician can view the temperature (heat) exposure data and assess what degree of further temperature exposure is acceptable in view of clinical considerations including the patient's disease state, their current symptoms, adjacent anatomical sites of importance, and the like. Using such information, the clinician can make decisions such as how aggressive further treatment should be. While embodiments herein can be applied to any type of patient tissue, in particular embodiments systems herein are adapted for use in treating tissue inside the head. Such tissue may be particularly susceptible to the effects of heat exposure and/or be liable to create more substantial side effects upon being impacted by heat exposure. As such, tracking temperature data in the head can be particularly important.

Temperature data herein can include, but is not limited to, tissue temperatures, core temperature values, an index or baseline temperature, temperatures at the site of electrodes for delivery of electrical stimulation, temperature at sites adjacent to electrodes and/or leads that such electrodes may be disposed on, and/or temperatures at various locations within a zone of exposure to electrical stimulation.

In various embodiments, temperature data can be indexed (or otherwise accounted for) to the sensor environment. For example, a temperature sensor positioned directly under an electrode can produce temperature data that is largely directly reflective of the temperature of the tissue interfacing with that electrode. An otherwise similar sensor that is near an electrode, but not directly under it, can produce temperature data that indirectly reflects a temperature of tissue interfacing with the electrode. As such, systems herein can be configured to index temperature data (or otherwise process or operate on the data) based on parameters such as direct or indirect measurement, tissue type, and/or electrode proximity (proximity of the temperature sensor to the electrode), as well as other parameters.

In some embodiments, the activity state and/or operational history of an electrode can be used as parameters in processing temperature data herein. For example, an electrode used to provide electrical field stimulation that has been turned off (e.g., no longer providing electrical field stimulation) for a sufficiently long period of time may reach a steady-state temperature (steady state “off” temperature) reflecting core body temperature and/or an index or baseline temperature of tissue. However, if the electrode has not been turned off for a sufficiently long period of time, then the temperature data may reflect a point somewhere in a transitional period between an “on” temperature and the steady-state off temperature. Therefore, the system can be configured to use the activity state and/or operational history of the electrode(s) when evaluating temperature data from temperature sensors associated with the same (directly or indirectly). For example, in some embodiments, temperature data reflecting a transition period after an electrode is turned off or on can be discarded as ambiguous data. In some embodiments, the transition period can be assumed to last a predetermined period of time. In other embodiments, the temperature can be evaluated and tracked to determine when a steady-state temperature has been reached after an operational event (such as turning off or on) of the electrode.

Embodiments of systems herein can also be configured to modulate a therapy parameter based on the temperature data. The therapy parameter can include at least one selected from the group consisting of a field strength, a duty cycle, and a therapy vector. In some embodiments, the system is configured to modulate a therapy parameter to limit the amount of time that the tissue is exposed to temperatures above a threshold level. In some embodiments, the system is configured to reduce a therapy parameter value (e.g., modulate a therapy value in a manner to reduce the amount of waste heat generated) to limit the amount of time that the tissue is exposed to temperatures above a threshold level over a specific time period and then later increase the therapy parameter value.

In various embodiments, modulation of therapy herein can be performed by the system as part of a closed loop thermal control system. In some embodiments, a closed loop thermal control system herein can operate using a thermal setpoint value or range. The thermal setpoint value can be a type of therapy parameter herein. The thermal setpoint value can be predetermined, static or dynamic, and/or can be set by a system user. The system can operate to maintain a temperature based on the thermal setpoint. In various embodiments, therapy parameters can be modulated in order to achieve a thermal setpoint temperature or a temperature falling within a thermal setpoint range. In addition, or in replacement, in some embodiments, the closed loop thermal setpoint value (specific temperature or range) itself can be changed, such as changed as a function of a temperature measurements herein.

The total heat exposure of tissue is not merely reflective of waste heat generated by systems producing electrical stimulation. Rather, the heat of the patient's body also contributes to total heat exposure. Further, the patient's body temperature fluctuates based on various conditions or cycles. As an example, a patient with a viral infection may have a fever which can include core and/or baseline temperature increases up to 3 degrees Celsius. As another example, during intense exercise the body's heat production may exceed 1000 W and body temperature may increase by up to 3 degrees Celsius. Conversely, a patient that is sleeping may have a core body temperature or reference temperature that is reduced. Other conditions and cycles (circadian and hormonal) may also cause characteristic increases or decreases of core body temperature or a reference temperature. Embodiments of systems herein can not only track such temperature fluctuations, but can also maximize electrical stimulation therapy in view of the same while not exceeding threshold for heat exposure of the tissue. For example, in some embodiments therapy parameter values can be reduced when core body temperature and/or a reference temperature is increased. As a specific example, therapy parameter values can be reduced (e.g., modulate a therapy value in a manner to reduce the amount of waste heat generated) when the patient has a fever and/or when the patient is exercising. Conversely, therapy parameter values can be increased when core body temperature and/or a reference temperature is decreased. For example, therapy parameter values can be increased when the patient is sleeping. As such, in various embodiments herein the system can be configured to execute activity/condition-based therapy modulation.

In some embodiments, temporal aspects of detected activity can be used by the system in activity/condition-based therapy modulation. For example, while exercise can increase body temperature, there is typically a delay between beginning exercise and the onset of increased bodily temperatures. Similarly, there is typically a delay between ceasing exercise and observing temperatures returning to pre-exercise levels. As such, in various embodiments, systems herein can take into account temporal aspects when executing activity or condition-based therapy modulation. For example, in some embodiments the system can take into account changes in temperature occurring at a predicted future time point based on current observations (data) related to patient activities and/or patient condition when modulating therapy parameters. In some embodiments, this can take the form of proactively making changes in therapy parameters based on temporal aspects of detected activity/condition. Conversely, in some embodiments, this can take the form of delaying (or offsetting in time) changes in therapy parameters based on temporal aspects of detected activity/condition. For example, the system can be configured to delay making changes in therapy parameters made to reduce heat generation in response to detected exercise.

Some types of temperature fluctuations may be unique to an individual. Specifically, the magnitude of temperature change in response to certain events can be unique to an individual. For example, some individuals may regularly experience a core or reference temperature drop during sleep of a first magnitude. In contrast, other individuals may regularly experience a core or reference temperature drop during sleep of a different magnitude. In addition, based on differences in the exact implantation sites of electrodes, some individuals may experience an increase in temperature at a tissue site of a first magnitude for a given intensity level of therapy whereas other individuals may experience an increase in temperature of a different magnitude. Understanding how a given individual will likely experience temperature fluctuations can be extremely valuable to characterize and/or provide to a clinician and/or to automatically adjust therapy intensity by the system itself. In some embodiments, temperature data of an individual can be collected and recorded over a learning phase and then processed by the system and/or computing resources in data communication therewith in order to characterize the individual's unique temperature fluctuation response to electrical stimulation therapy and/or events such as fever, exercise, sleep and the like and/or produce a model of the same. Such characterizations and/or the model can then be used by the system and/or provided to a clinician in order to plan/program therapy to be delivered to the patient by the system. For example, such characterizations and/or the model can then be used by the system and/or provided to a clinician and used to accurately predict temperatures of tissues resulting from different intensities of electrical stimulation therapy as administered at various times. These and other embodiments will now be illustrated with respect to some examples shown and described in the figures.

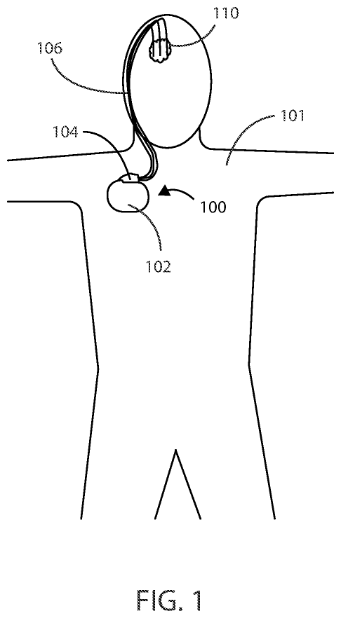

Referring now to FIG. 1, a schematic view of a medical device 100 implanted in a patient 101 is shown in accordance with the embodiments herein. In FIG. 1, the patient 101 has the medical device 100 implanted entirely within the body of the patient 101 at or near a tumor resection site 110. It will be appreciated that while many embodiments herein disclose a tumor resection site 110, area 110 may alternatively represent a cancerous or non-cancerous tumor site, zone, or cavity. Various implant sites can be used including areas such as in the limbs, the upper torso, the abdominal area, the head, and the like. In some embodiments, the medical device can be at least partially implanted within the body of the patient at or near the site of the cancerous tumor. In various embodiments, the medical device can be entirely external to the patient. In some embodiments, the medical device can be partially external to the patient. In some embodiments, the medical device can be partially implanted and partially external to the body of a patient. In other embodiments, a partially implanted medical device can include a transcutaneous connection between components disposed internal to the body and external to the body. A partially or fully implanted medical device can wirelessly communicate with a partially or fully external portion of a medical device over a wireless connection.

In some embodiments, a portion of the medical device can be entirely implanted, and a portion of the medical device can be entirely external. For example, in some embodiments, one or more electrodes or leads can be entirely implanted within the body, whereas the portion of the medical device that generates an electric field, such as an electric field generator, can be entirely external to the body. It will be appreciated that in some embodiments described herein, the electric field generators described can include many of the same components and can be configured to perform many of the same functions, as a pulse generator. In embodiments where a portion of a medical device is entirely implanted, and a portion of the medical device is entirely external, the portion of the medical device that is entirely external can communicate wirelessly with the portion of the medical device that is entirely internal. However, in other embodiments a wired connection can be used.

The medical device 100 can include a housing 102 and a header 104 coupled to the housing 102. Various materials can be used. However, in some embodiments, the housing 102 can be formed of a material such as a metal, ceramic, polymer, composite, or the like. In some embodiments, the housing 102, or one or more portions thereof, can be formed of titanium. The header 104 can be formed of various materials, but in some embodiments the header 104 can be formed of a translucent polymer such as an epoxy material. In some embodiments the header 104 can be hollow. In other embodiments the header 104 can be filled with components and/or structural materials such as epoxy or another material such that it is non-hollow.

In some embodiments where a portion of the medical device 100 is partially external, the header 104 and housing 102 can be surrounded by a protective casing made of durable polymeric material. In other embodiments, where a portion of the medical device 100 is partially external, the header 104 and housing 102 can be surrounded by a protective casing made of a combination of polymeric material, metallic material, and/or glass material.

Header 104 can be coupled to one or more leads, such as leads 106. The header 104 can serve to provide fixation of the proximal end of one or more leads 106 and electrically couple the one or more leads 106 to one or more components within the housing 102.

The one or more leads 106 can include one or more electrodes (not shown in this view) disposed along the length of the leads 106. In some embodiments, electrodes can include electric field generating electrodes, also referred to herein as “supply electrodes.” In some embodiments electrodes can include electric field sensing electrodes, also referred to herein as “sensing electrodes.” In some embodiments, leads 106 can include both supply electrodes and sensing electrodes. In other embodiments, leads 106 can include any number of electrodes that are both supply electrodes and sensing electrodes.

The one or more leads 106 can also include one or more temperature sensors (not shown in this view) disposed along the length of the leads 106. Temperature sensors herein can include, but are not limited to, various types of optical and electrical temperature sensors. Temperature sensors herein can include contact-type temperature sensors and non-contact type temperature sensors. Optical temperature sensors herein can include infrared optical temperature sensors. Some optical temperature sensors can measure temperature at a distance such as a distance of millimeters or centimeters. Thus, even where temperature sensors are mounted along a lead 106, temperature can be measured at a distance therefrom. Exemplary electrical temperature sensors can include, but are not limited to, thermistors, resistive temperature detectors, thermocouples, semiconductor based temperature sensors, and the like.

It will be appreciated that while many embodiments of medical devices herein are designed to function with leads, leadless medical devices that generate electrical fields are also contemplated herein. In some embodiments, the electrodes can be tip electrodes on the most distal end of the leads 106.

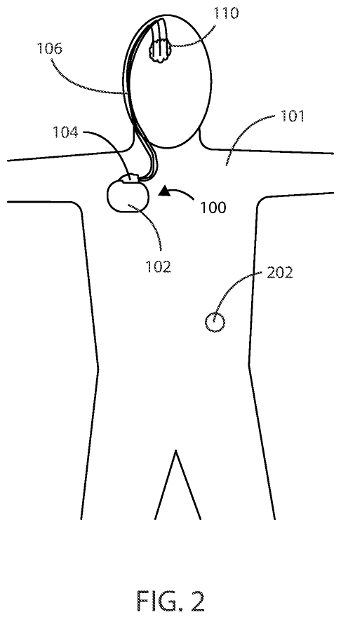

In some embodiments, the medical device system can further include a temperature sensor disposed remotely from the medical device. A remote temperature sensor can provide temperature data in addition to or in replace of temperature sensors in other areas such as along the leads 106. In some embodiments, a remote temperature sensor can be used to gather a core or reference temperature of the patient into which the system is implanted. Referring now to FIG. 2, a schematic view of a medical device 100 implanted in a patient 101 is shown in accordance with various embodiments herein. The medical device 100 implanted within the patient 101 includes implantable components such as a housing 102, a header 104, and leads 106. In this view, the patient 101 is shown to include a tumor resection site 110. The leads 106 can be implanted within the patient 101 in order to interface with the tumor resection site 110 and/or be adjacent to the tumor resection site 110 such that electrical fields generated through the leads 106 can interface with the tumor resection site 110.

In some embodiments, a temperature sensor 202 can be implanted within the patient 101 separately from the medical device 100. In various embodiments, the temperature sensor 202 can be entirely implanted within the patient's abdomen. The temperature sensor 202 can wirelessly communicate with the medical device 100 to provide reference or core body temperature data of the patient 101. In some embodiments, the temperature sensor 202 can send a measured or estimated reference or core body temperature of the patient 101 to the medical device 100 along with the associated time stamp. It will be appreciated that while some embodiments disclose a single temperature sensor, a greater or lesser number of temperature sensors can be used. By way of example, one, two, three, four, or more temperatures sensors are contemplated herein. Additionally, in some embodiments, temperature sensors can be positioned internally or externally on a patient's body. For example, in various embodiments, a temperature sensor can be positioned proximal to the cancer therapy stimulation leads, discussed in greater detail below. In some embodiments, a temperature sensor can be entirely implanted within the patient's abdomen, chest, groin, pelvis, or hip, and the like. In other embodiments, a temperature sensor can be positioned externally on a patient's arm, leg, abdomen, chest, and the like.

In some embodiments, in response to receiving a patient's reference or core body temperature, medical device 100 can modulate one or more of the therapy parameters. This can include modulating the applied field strength, pattern or timing of the duty cycle, waveform, or therapy vector such as therapy stimulation vector or vector switching pattern. In some embodiments, in response to receiving a patient's reference or core body temperature, medical device 100 can make suggestions (such as to a clinician) regarding a recommended level of therapy intensity and/or a change in a level of therapy intensity in view of the reference and/or core body temperature. For example, if the reference and/or core body temperature decreases or is below a threshold value, the suggestion may be to increase the intensity of the electrical field therapy (e.g., change parameters of the therapy to increase the intensity and/or energy output thereof). Conversely, if the reference and/or core increases or is above a threshold value, the suggestion may be to decrease the intensity of the electrical field therapy (e.g., change parameters of the therapy to decrease the intensity and/or energy output thereof).

In various embodiments, the medical device 100 can modulate a therapy parameter in response to a patient's reference or core body temperature exceeding a threshold level. Alternatively, the medical device 100 can modulate a therapy parameter after a set period of time. In other embodiments, the medical device 100 can modulate a therapy parameter in response to a patient's reference or core body temperature exceeding a threshold level for a set period of time. For example, the medical device can track the amount of time the patient's tissue is exposed to temperatures above an identified temperature. It is contemplated herein that the higher the set threshold temperature is, the lower the amount of time a patient can be exposed to that temperature and vice versa. For example, if a threshold temperature level is 39 degrees Celsius, a patient could be exposed to that temperature for X minutes before a therapy parameter is modified. Alternatively, if a threshold temperature level is 44 degrees Celsius, a patient could be exposed to that temperature for Y seconds (e.g., a lesser amount of time than X minutes) before a therapy parameter is modified.

In some embodiments, a clinician can set the threshold level. In other embodiments, the medical device 100 can have preprogrammed threshold levels. It is understood that exposure to temperatures above body temperature can impact or otherwise damage healthy tissue depending on the length of time the tissue is exposed to the temperature. In other words, the impact on tissue is a matter of both temperature and the amount of time at that temperature. Thus, it will be appreciated that the medical device is configured to modulate a therapy parameter in order to limit the amount of time a patient's tissue is exposed to temperatures above the set threshold level. Additionally, it will be appreciated that the medical device is configured to regulate the threshold temperature level to contain maximal therapy dosing.

The medical device 100 can determine if a threshold level has been reached using a variety of means. In some embodiments, the medical device 100 can determine when a threshold temperature or set time period has been reached. Alternatively, the medical device 100 can calculate an average tissue or body temperature by adding all measured or estimated tissue temperatures over a period time. In other embodiments, the medical device 100 can calculate an average tissue or body temperature by calculating the equivalent minutes of the tissue at a predetermined temperature over a set period of time.

In various embodiments, the medical device 100 can modulate a therapy parameter for a specified period of time and then later increase or decrease the therapy parameter value based on the patient's reference or core temperature. In some embodiments, the medical device 100 can increase the intensity of the therapy if the reference or core body temperature is lower than a threshold temperature level. For example, if a patient is asleep and the reference or core body temperature is below the threshold level a therapy parameter can be increased. In other embodiments, the medical device 100 can decrease the intensity of the therapy if the reference or core body temperature is elevated. For example, if a patient has a fever and the reference or core body temperature is elevated above a normal core body temperature or above a threshold temperature level a therapy parameter can be changed to decrease the intensity of the therapy.

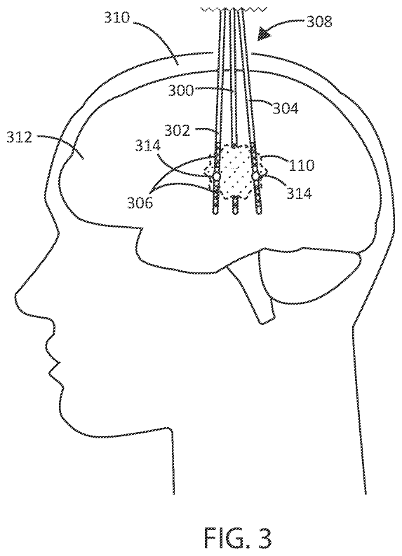

In some embodiments, the medical device 100 can include a plurality of stimulation leads implanted at or near a site of a cancerous tumor or tumor resection. Referring now to FIG. 3, a schematic view of a placement of various cancer therapy stimulation leads 300, 302, 304 in a region of a tumor resection site 110 or tumor is shown in accordance with various embodiments herein. In the example of FIG. 3, temperature sensors 314 are disposed on the stimulation leads. The temperature sensors 314 can be of any type described elsewhere herein. In the embodiment shown in FIG. 3, each cancer therapy stimulation lead includes two electric field generating electrodes 306 disposed along a length of the cancer therapy stimulation leads. Each cancer therapy stimulation lead includes a proximal and a distal electric field generating electrode. It will be appreciated that each cancer therapy stimulation lead can include one or more electric field generating electrodes. Exemplary cancer therapy stimulation leads are disclosed in commonly owned U.S. Pat. Appl. No. 63/298,528, the content of which is hereby incorporated by reference in its entirety. The cancer therapy stimulation leads and the electric field generating electrodes disposed thereon are discussed in more detail below.

The side view shown in FIG. 3 also includes the placement of cancer therapy stimulation leads 300, 302, and 304 around the tumor resection site 110 and in position within a burr hole 308 entry point on the patient's skull 310 within the patient's brain 312. It will be appreciated that in some embodiments one burr hole can be used with one or more (e.g., one, two, three, or more) leads and/or electrodes. In some embodiments, multiple burr holes can be used each with one or more (e.g., one, two, three, or more) leads and/or electrodes.

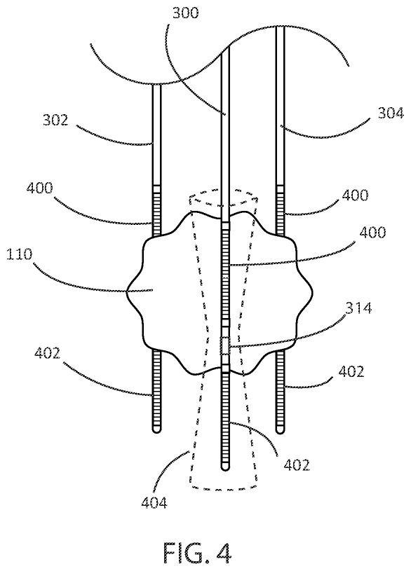

Referring now to FIG. 4, a schematic view of a placement of various cancer therapy stimulation leads 300, 302, 304 in a region of a tumor resection site 110 is shown in accordance with various embodiments herein. Each of the cancer therapy stimulation leads 300, 302, and 304 include two electric field generating electrodes (e.g., a proximal electrode 400 and a distal electrode 402) and a temperature sensor 314. However, it will be appreciated that in some embodiments a greater or lesser number of electrodes and/or temperature sensors can be used. In some embodiments, each of the cancer therapy stimulation leads 300, 302, and 304 can include a different number of electrodes from one another. By way of example, each cancer therapy stimulation lead can include one electrode, two electrodes, or a combination thereof. The cancer therapy stimulation leads 300, 302, and 304 are arranged to be in proximity to the cancerous tumor or tumor resection site 110. The electric fields can be visualized through the coil electrodes along various stimulation vectors (a pathway through the tissue/tumor interconnecting two different electrodes).

Each cancer therapy stimulation lead 300, 302, and 304 can generate an electric field strength zone 404 about the longitudinal axis of the cancer therapy stimulation leads. The electric field strength zone 404 can be shaped like an hourglass, a cylinder, and the like. The electric field strength zone of the cancer therapy stimulation leads can center around the electric field generating electrodes 400 and 402.

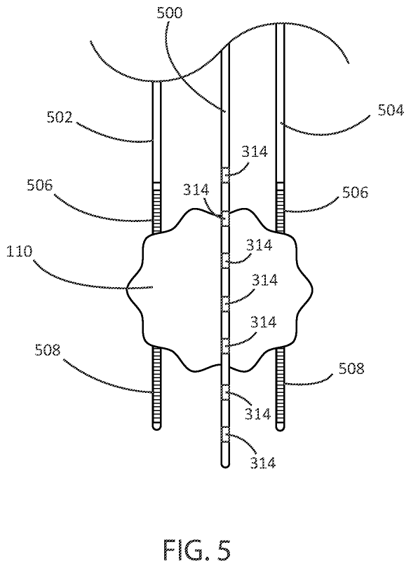

In some embodiments, temperature sensors can be disposed on stimulation leads along with electrodes. However, in some embodiments temperature sensors can be disposed on separate leads other than stimulation leads. Referring now to FIG. 5, a schematic view of a placement of various cancer therapy stimulation leads 502 and 504 and a temperature sensor lead 500 is shown in accordance with various embodiments herein. The two-cancer therapy stimulation leads 502 and 504 are arranged in proximity to the tumor resection site 110. Each of the cancer therapy stimulation leads 502 and 504 include two electric field generating electrodes (e.g., a proximal electrode 506 and a distal electrode 508). It will be appreciated the cancer therapy stimulation leads may be modified in any manner similar to that as described above with respect to FIG. 4.

In addition to the cancer therapy stimulation leads 302 and 304, FIG. 5 includes a temperature sensor lead 500 arranged in proximity to the tumor resection site 110 and the cancer therapy stimulation leads 502 and 504. The temperature sensor lead 500 can include a plurality of temperature sensors 314 thereon. The temperature sensor lead 500 can be used to measure the tissue temperature proximate to the treatment site. In some embodiments, the temperature sensor lead 500 can send a measured or estimated tissue temperature of the patient 101 to the medical device 100 along with the associated time stamp. It will be appreciated the medical device 100 can modulate a therapy parameter based on the temperature of the tissue proximate to the treatment site in a similar manner for similar reasons as described above with respect to FIG. 2.

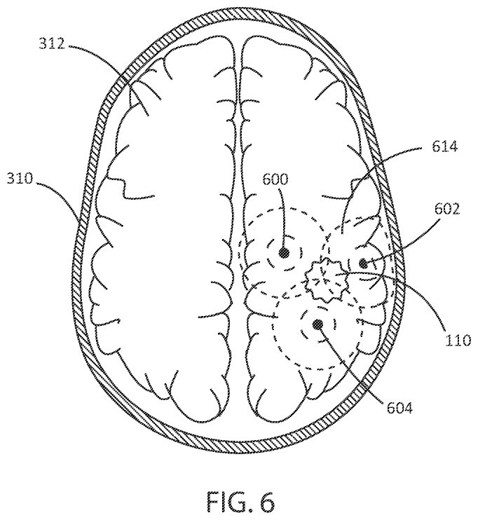

Referring now to FIG. 6, a top view of a placement of various cancer therapy stimulation leads 600, 602, 604 in a region of a tumor resection site 110 is shown in accordance with various embodiments herein. As shown in FIG. 6, the tumor resection site 110 can be located within a patient's brain 312. In some embodiments, the cancer therapy stimulation leads 600, 602, and 604 can be positioned in a region containing the tumor resection site 110. In some embodiments, the cancer therapy stimulation leads 600, 602, and 604 can be positioned about a center point of the tumor resection site 110. In other embodiments, the cancer therapy stimulation leads 600, 602, and 604 can be positioned about a non-centered point of the tumor resection site 110. In some embodiments, the cancer therapy stimulation leads 600, 602, and 604 can be positioned about the circumference of the tumor resection site 110.

In various embodiments, the cancer therapy stimulation leads 600, 602, and 604 can each include an elevated temperature zone 614 (indicated by the dotted lines surrounding each cancer therapy stimulation lead) described in greater detail below.

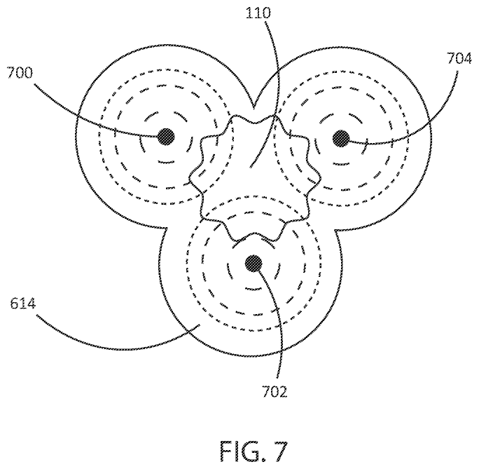

In various embodiments, the cancer therapy stimulation leads herein can be placed at from 70 degrees to 120 degrees apart from one another relative to the center point of the cancerous tumor. In various embodiments, the cancer therapy stimulation therapy leads can be positioned apart from one another around a cancerous tumor at from greater than or equal to 70 degrees, 80 degrees, 90 degrees, 100 degrees, 110 degrees, 120 degrees, 130 degrees, 140 degrees, 150 degrees, 160 degrees, 170 degrees, or 180 degrees, about a cancerous tumor relative to one another, or can be an amount falling within a range between any of the foregoing. By way of example, the cancer therapy stimulation leads 600, 602, 604 in FIG. 6 are shown to be positioned at 120 degrees, about the center point of the tumor resection site 110. In some embodiments the cancer therapy stimulation leads can be spaced equally about center point of the cancerous tumor. In other embodiments, the cancer therapy stimulation leads can be spaced unequally about the center point of the cancerous tumor. In various embodiments, one or more cancer therapy stimulation leads can be positioned within the cancerous tumor. FIG. 7 includes exemplary cancer therapy stimulation lead placements where various numbers of cancer therapy stimulation leads are shown positioned within a region of a cancerous tumor or a tumor resection site. FIG. 7 is specifically a top view of a placement of three cancer therapy stimulation leads 700, 702, 704 in a region of a tumor resection site 110 is shown in accordance with various embodiments herein. Cancer therapy stimulation leads 700, 702, and 704 can be positioned around a center point of the cancerous tumor or tumor resection site 110 at approximately 120 degrees apart from each other. Each cancer therapy stimulation lead can be modeled to include an elevated temperature zone 604 around the circumference of the lead. It will be appreciated that the temperature of each elevated temperature zone can decrease in a radial direction away from the center of the cancer therapy stimulation lead 204.

### Methods

Many different methods are contemplated herein, including, but not limited to, methods of making, methods of using, methods of treating cancer, methods of treating a patient, and the like. Aspects of system/device operation described elsewhere herein can be performed as operations of one or more methods in accordance with various embodiments herein.

In various embodiments, operations described herein and method steps can be performed as part of a computer-implemented method executed by one or more processors of one or more computing devices. In various embodiments, operations described herein and method steps can be implemented instructions stored on a non-transitory, computer-readable medium that, when executed by one or more processors, cause a system to execute the operations and/or steps.

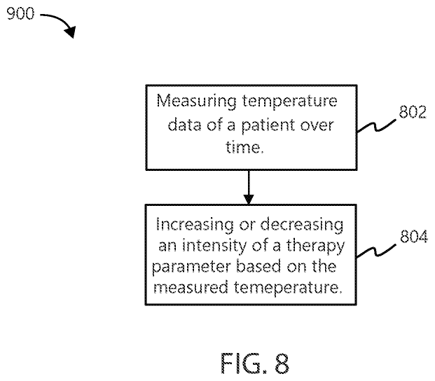

Referring now to FIG. 8, in an embodiment, a method 800 for modulating a therapy parameter is included. The method can include measuring temperature data of a patient over time 802. In various embodiments, one or more temperature sensors can be positioned on the implantable system thereby allowing the temperature of a patient to be taken at the treatment site. In other embodiments, one or more temperature sensors can be separate from, but wirelessly connected to, the implantable system. For example, one or more temperature sensors can be positioned in the patient's abdomen. This can allow a reference or core temperature of the patient to be taken. In some embodiments, temperature data can be taken at set intervals. For example, a patient's temperature can be taken every 5 minutes, 10 minutes, 15 minutes, 20 minutes, 25 minutes, or 30 minutes or more, or can fall within a range between any of the foregoing. In some embodiments, a patient's temperature can be continuously monitored.

The method can further include increasing or decreasing an intensity of a therapy parameter based on the measured temperature 804. This can include modulating the applied field strength, the pattern or timing of the duty cycle, waveform, or therapy vector such as therapy stimulation vector or vector switching pattern.

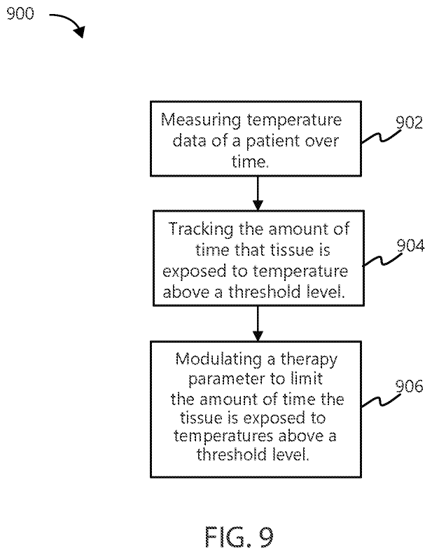

In some embodiments, a method for modulating a therapy parameter is included. FIG. 9 shows a flow chart showing a method 900 of modulating a therapy parameter based on tracking temperature data of a patient over time according to an embodiment.

In some embodiments, the method 900 can include measuring temperature data of a patient over time 902 in a manner similar to that as described with respect to FIG. 8.

In various embodiments, the method can include tracking the amount of time that tissue is exposed to temperature above a threshold level 904. The amount of time and the threshold level can both be set by a patient's clinician. In some embodiments, the clinician may set shorter amounts of time and higher threshold temperature levels. For example, the clinician may set the implantable device to modulate a therapy parameter after five minutes of a temperature above 40 degrees Celsius (as merely one example, other times and temperatures are contemplated herein). In other embodiments, the clinician may increase the amount of time allowable for lower threshold temperature levels. For example, the clinician may set the implantable device to modulate a therapy parameter after 20 minutes of a temperature above 38 degrees Celsius (as merely another example, other times and temperatures are contemplated herein).

The method can further include modulating a therapy parameter to limit the amount of time the tissue is exposed to temperatures above a threshold level 906 in a manner similar to that as described with respect to FIG. 8.

In an embodiment, a method of providing electrical stimulation cancer therapy is included herein. The method can include generating an electrical output for one or more electrodes to create one or more electric fields, measuring temperature and/or recording temperature data of a patient over time, and processing the temperature data and modulating a therapy parameter based on the temperature data.

In an embodiment, the method can further include using activity state data of the one or more electrodes and/or operational history of the one or more electrodes when processing the temperature data.

In an embodiment, the method can further include processing the temperature data and modulate the therapy parameter based on the temperature data as part of a closed loop control system.

In an embodiment, the method can further include indexing the temperature data based on one or more of direct or indirect measurement, tissue type, and electrode proximity.

In an embodiment, the method can further include tracking the amount of time that tissue is exposed to temperatures above a threshold level.

In an embodiment, the method can further include modulating a therapy parameter to limit the amount of time that the tissue is exposed to temperatures above a threshold level.

In an embodiment, the method can further include reducing a therapy parameter value to limit the amount of time that the tissue is exposed to temperatures above a threshold level over a specific time period and then later increasing the therapy parameter value.

In an embodiment, the method can further include issuing an alert if the amount of time that tissue is exposed to temperatures above a threshold level exceeds a predetermined amount of time.

In an embodiment, the method can further include increasing an intensity of the therapy at times when a core body temperature or a reference body temperature is lower than a threshold temperature.

In an embodiment, the method can further include increasing an intensity of the therapy parameter at times when the patient is asleep.

In an embodiment, the method can further include decreasing an intensity of the therapy parameter at times when the patient has an elevated core body temperature or reference body temperature.

In an embodiment, the method can further include decreasing an intensity of the therapy parameter at times when the patient has a fever.

In an embodiment, the method can further include measuring or estimate a core body temperature or a reference body temperature when therapy is turned off or paused.

In an embodiment, the method can further include measuring or estimate a core body temperature or reference body temperature of the patient.

In an embodiment, the method can further include: measuring and/or receive a core body temperature or a reference body temperature of the patient, and increasing an intensity of the therapy parameter at times when the core body temperature or reference body temperature is lower than a threshold temperature.

In an embodiment, the method can further include measuring a temperature at a site along a stimulation lead. In an embodiment, the method can further include measuring a temperature at a site offset from a surface of an electrode. In an embodiment, the method can further include measuring a temperature at a site underneath a surface of an electrode.

In an embodiment, the method can further include estimating physical activity based on a signal from an accelerometer and modulating a therapy parameter based on the same. In an embodiment of the method, estimating physical activity based on a signal from an accelerometer and modulating a therapy parameter based on the same comprises modulating the therapy parameter based on estimated physical activity in advance of measured temperature changes.

In an embodiment, the method can further include regulating therapy zone temperature limits to contain maximal therapy dose.

### Alerts

In an embodiment, the implantable system can include an alert system that sends one or more alerts to a system user such as a clinician or other care provider. For example, the alert system can warn a user if the amount of time that tissue is exposed to temperatures above a threshold level exceeds a predetermined amount of time. Additionally, the alert system can warn a user when a therapy parameter is modulated. For example, the alert system can warn a user when a therapy parameter value is increased or decreased.

The alerts can be sent to a variety of individuals. For example, the alerts can be sent wirelessly to a patient, a clinician, a care provider, or the like. Additionally, the alert system can send alerts to a variety of devices. For example, the alerts can be sent to a computer, phone, smartwatch, or medical record system. The alert system can warn a user using a variety of means. For example, the alert system could provide a visual pop-up alert on a device and/or the alert system could provide a sound alert.

### Cancer Therapy Stimulation Leads

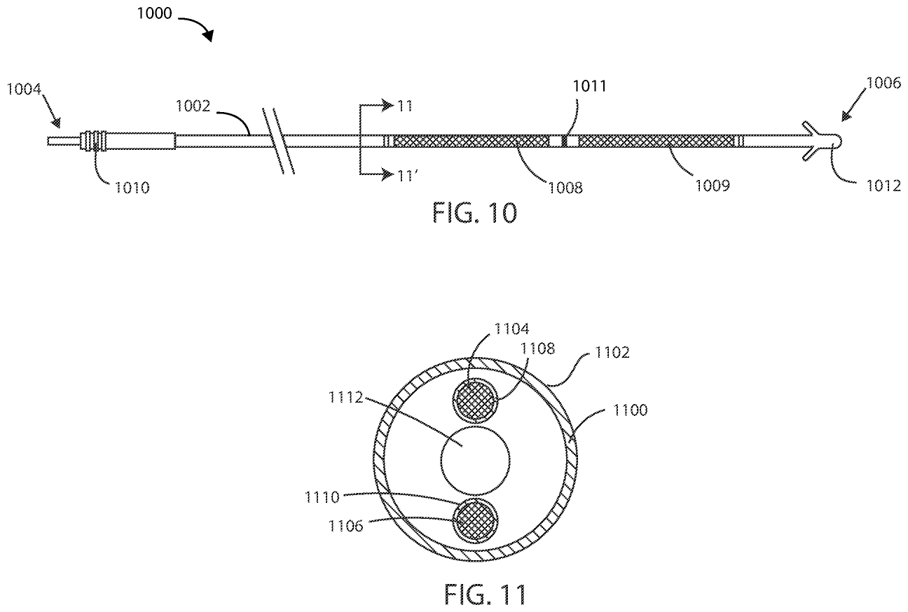

Referring now to FIG. 10, a schematic view of an exemplary cancer therapy stimulation lead 1000 is shown in accordance with various embodiments herein. The cancer therapy stimulation lead 1000 can include a lead body 1002 with a proximal end 1004 and a distal end 1006. In this example, a first electrode 1008 and a second electrode 1009 are coupled to the lead body 1002, as positioned near a distal end 1006 thereof. In some embodiments, the electrodes 1008, 1009 can include electric field generating electrodes. In various embodiments, the electrodes 1008, 1009 can include electric field sensing electrodes. The electrodes 1008, 1009 can be internally connected or internally independent. In an example where the electrodes 1008, 1009 are independent, the system herein can model each as an independent field and heat source. The lead body 1002 can define a lumen. The electrodes 1008, 1009 can include various conductive materials such as platinum, silver, gold, iridium, titanium, and various alloys. In some embodiments, the cancer therapy stimulation lead 1000 includes more than two electrodes.

The cancer therapy stimulation lead 1000 can include one or more therapy zone temperature sensors disposed along a length of the cancer therapy stimulation lead. In this example, a therapy zone temperature sensor 1011 is positioned between the first electrode 1008 and the second electrode 1009. However, the therapy zone temperature sensor 1011 can also be positioned at various other points along or in the lead. In some embodiments, the therapy zone temperature sensor 1011 can be positioned in the lead directly beneath an electrode. The therapy zone temperature sensor 1011 can include an optical or electrical thermal sensor. For example, the therapy zone temperature sensor can include a thermistor. The therapy zone temperature sensor 1011 can be used to measure the thermal heating about the cancer therapy stimulation lead to provide feedback to a clinician about the local thermal heating zone around the lead and provide a tissue temperature of the treatment site to the medical device.

In various embodiments, the therapy zone temperature sensor 1011 can provide a tissue temperature at a site offset from a surface of the electrodes 1008, 1009. If a tissue temperature of a site offset from the electrodes 1008, 1009 is measured, the medical device can compensate for the offset when measuring or estimating the temperature of the tissue. In some embodiments, the therapy zone temperature sensor 1011 can measure or estimate the reference or core body temperature of the patient when the therapy is turned off or paused. While not intending to be bound by theory, in some scenarios it can be easier to get an accurate measurement of a reference or core body temperature when therapy is turned off or paused. In some embodiments, therapy zone temperature sensor data can be recorded and relayed to the clinician, patient, care provider, and/or medical record system.

The cancer therapy stimulation lead 1000 can further include a terminal pin 1010 for connecting the cancer therapy stimulation lead 1000 to a medical device, such as a cancer treatment device. The terminal pin 1010 can be compatible with various standards for lead-header interface design including the DF-1, VS-1, IS-1, LV-1 and IS-4 standards, amongst other standards.

In some embodiments, the cancer therapy stimulation lead 1000 can further include a fixation element 1012, such as an element that can adhere to a portion of the subject's body to maintain the position of the cancer therapy stimulation lead 1000 and/or the electrodes 1008. In various embodiments, the fixation element 1012 can be disposed along the distal end 1006 of the cancer therapy stimulation lead 1000. However, in some embodiments a fixation element 1012 is omitted.

Referring now to FIG. 11, a cross-sectional schematic view of a cancer therapy stimulation lead 1000 as taken along line 11-11′ of FIG. 10 is shown in accordance with various embodiments herein. The cancer therapy stimulation lead 1000 can include an outer layer 1100 with an outer surface 1102. The outer layer 1100 can be flexible and can be configured to protect other components disposed within the lumen of the outer layer 1100. In some embodiments, the outer layer 1100 can be circular in cross-section. In some embodiments, the outer layer 1100 includes a dielectric material and/or an insulator. In some embodiments, the outer layer 1100 can include various biocompatible materials such as polysiloxanes, polyethylenes, polyamides, polyurethane and the like.

In various embodiments, the cancer therapy stimulation lead 1000 can include one or more conductors, such as a first conductor 1104 and a second conductor 1106. In some embodiments, the first conductor 1104 and the second conductor 1106 can be disposed within the lumen of the outer layer 1100. The first conductor 1104 and a second conductor 1106 can be configured to provide electrical communication between an electrode 1008 and the proximal end 1004 of the cancer therapy stimulation lead 1000. The first conductor 1104 and a second conductor 1106 can include various materials including copper, aluminum, silver, gold, and various alloys such as tantalum/platinum, MP35N and the like. An insulator 1108 and 1110 can surround the first conductor 1104 and a second conductor 1106. The insulators 1108 and 1110 can include various materials such as electrically insulating polymers.

In some embodiments, each of the electrodes 1008 can have individual first conductors 1104 and second conductors 1106 to electrically couple the electrode 1008 to the proximal end 1004 of the cancer therapy stimulation lead 1000. However, in some embodiments, each of the electrodes 1008 only connects to a single conductor to electrically couple the electrode 1008 to the proximal end 1004 of the cancer therapy stimulation lead 1000. In some embodiments, the first conductor 1104 and a second conductor 1106 can be configured as a coil or a cable. Multiple conductors can be disposed within the lumen of the outer layer 1100. For example, a separate conductor or set of conductors can be in communication with each electrode disposed along the lead. In various embodiments, a first conductor 1104 and a second conductor 1106 can form a part of an electrical circuit by which the electric fields from the electric field generating circuit are delivered to the site of the cancerous tissue. Many more conductors than are shown in FIG. 11 can be included within embodiments herein. For example, the cancer therapy stimulation lead 1000 can include 1, 2, 3, 4, 5, 6, 7, 8, 10, 15 or 20 or more conductors, or any number of conductors falling within a range between any of the foregoing.

In some embodiments, the cancer therapy stimulation lead 1000 can include a central channel 1112. The central channel 1112 can be configured for a guide wire, or other implanting device, to pass through, such as to aid in implanting the cancer therapy stimulation lead 1000 and electrodes 1008. In some cases, additional channels (not shown) are disposed within the cancer therapy stimulation lead 1000.

### Medical Device Components

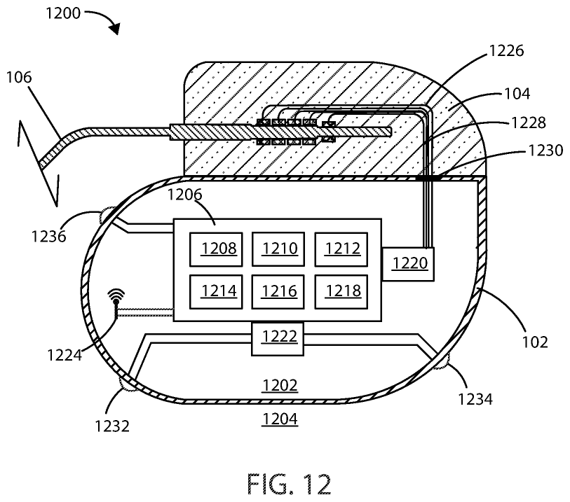

Referring now to FIG. 12, a schematic cross-sectional view of medical device 1200 is shown in accordance with various embodiments herein. The housing 102 can define an interior volume 1202 that can be hollow and that in some embodiments is hermetically sealed off from the area 1204 outside of medical device 1200. In other embodiments the housing 102 can be filled with components and/or structural materials such that it is non-hollow. The medical device 1200 can include control circuitry 1206, which can include various components 1208, 1210, 1212, 1214, 1216, and 1218 disposed within housing 102. In some embodiments, these components can be integrated and in other embodiments these components can be separate. In yet other embodiments, there can be a combination of both integrated and separate components. The medical device 1200 can also include an antenna 1224, to allow for unidirectional or bidirectional wireless data communication, such as with an external device or an external power supply. In some embodiments, the components of medical device 1200 can include an inductive energy receiver coil (not shown) communicatively coupled or attached thereto to facilitate transcutaneous recharging of the medical device via recharging circuitry.

The various components 1208, 1210, 1212, 1214, 1216, and 1218 of control circuitry 1206 can include, but are not limited to, a microprocessor, memory circuit (such as random access memory (RAM) and/or read only memory (ROM)), recorder circuitry, controller circuit, a telemetry circuit, a power supply circuit (such as a battery), a timing circuit, and an application specific integrated circuit (ASIC), a recharging circuit, amongst others. Control circuitry 1206 can be in communication with an electric field generating circuit 1220 that can be configured to generate electric current to create one or more fields. The electric field generating circuit 1220 can be integrated with the control circuitry 1206 or can be a separate component from control circuitry 1206. Control circuitry 1206 can be configured to control delivery of electric current from the electric field generating circuit 1220. In some embodiments, the electric field generating circuit 1220 can be present in a portion of the medical device that is external to the body.

In some embodiments, the control circuitry 1206 can be configured to direct the electric field generating circuit 1220 to deliver an electric field via leads 106 to the site of a cancerous tumor located within a bodily tissue. In other embodiments, the control circuitry 1206 can be configured to direct the electric field generating circuit 1220 to deliver an electric field via the housing 102 of medical device 1200 to the site of a cancerous tumor located within a bodily tissue. In other embodiments, the control circuitry 1206 can be configured to direct the electric field generating circuit 1220 to deliver an electric field between leads 106 and the housing 102 of medical device 1200. In some embodiments, one or more leads 106 can be in electrical communication with the electric field generating circuit 1220.

In some embodiments, various components within medical device 1200 can include a therapy output circuit, for example, an electric field sensing circuit 1222 configured to generate a signal corresponding to sensed electric fields. Electric field sensing circuit 1222 can be integrated with control circuitry 1206 or it can be separate from control circuitry 1206.

Sensing electrodes can be disposed on or adjacent to the housing of the medical device, on one or more leads connected to the housing, on a separate device implanted near or in the tumor, or any combination of these locations. In some embodiments, the electric field sensing circuit 1222 can include a first sensing electrode 1232 and a second sensing electrode 1234. In other embodiments, the housing 102 itself can serve as a sensing electrode for the electric field sensing circuit 1222. The electrodes 1232 and 1234 can be in communication with the electric field sensing circuit 1222. The electric field sensing circuit 1222 can measure the electrical potential difference (voltage) between the first electrode 1232 and the second electrode 1234. In some embodiments, the electric field sensing circuit 1222 can measure the electrical potential difference (voltage) between the first electrode 1232 or second electrode 1234, and an electrode disposed along the length of one or more leads 106. In some embodiments, the electric field sensing circuit can be configured to measure sensed electric fields and to record electric field strength in V/cm.

It will be appreciated that the electric field sensing circuit 1222 can additionally measure an electrical potential difference between the first electrode 1232 or the second electrode 1234 and the housing 102 itself. In other embodiments, the medical device can include a third electrode 1236, which can be an electric field sensing electrode or an electric field generating electrode. In some embodiments, one or more sensing electrodes can be disposed along lead 106 and can serve as additional locations for sensing an electric field. Many combinations can be imagined for measuring electrical potential difference between electrodes disposed along the length of one or more leads 106 and the housing 102 in accordance with the embodiments herein.

In some embodiments, the one or more leads 106 can be in electrical communication with the electric field generating circuit 1220. The one or more leads 106 can include one or more electrodes 306, as shown in FIG. 3. In some embodiments, various electrical conductors, such as electrical conductors 1226 and 1228, can pass from the header 104 through a feed-through structure 1230 and into the interior volume 1202 of medical device 1200. As such, the electrical conductors 1226 and 1228 can serve to provide electrical communication between the one or more leads 106 and control circuitry 1206 disposed within the interior volume 1202 of the housing 102.

In some embodiments, recorder circuitry can be configured to record the data produced by the electric field sensing circuit 1222 and record time stamps regarding the same. In some embodiments, the control circuitry 1206 can be hardwired to execute various functions, while in other embodiments the control circuitry 1206 can be directed to implement instructions executing on a microprocessor or other external computation device. A wireless communication interface can also be provided for communicating with external computation devices such as a programmer, a home-based unit, and/or a mobile unit (e.g., a cellular phone, personal computer, smart phone, tablet computer, smartwatch, and the like).

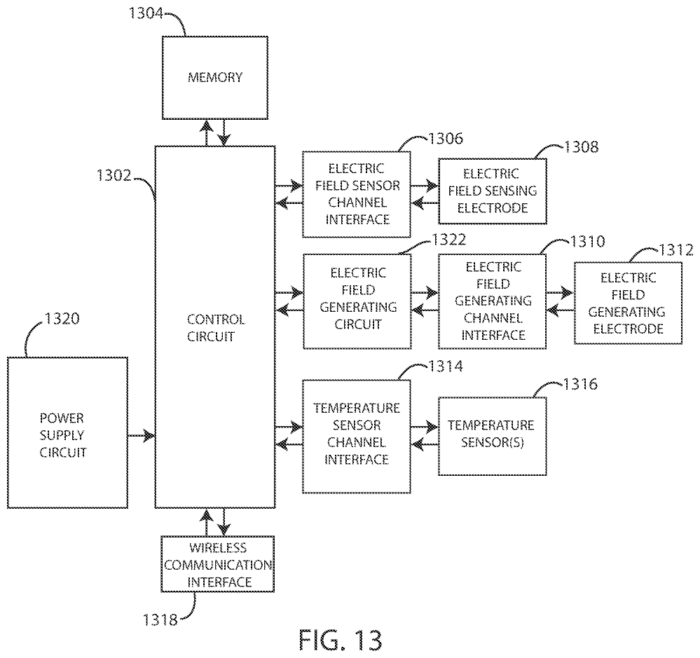

Elements of various embodiments of the medical devices described herein are shown in FIG. 13. However, it will be appreciated that some embodiments can include additional elements beyond those shown in FIG. 13. In addition, some embodiments may lack some elements shown in FIG. 13. The medical devices as embodied herein can gather information through one or more sensing channels and can output information through one or more field generating channels. A microprocessor 1302 can communicate with a memory 1304 via a bidirectional data bus. The memory 1304 can include read only memory (ROM) or random-access memory (RAM) for program storage and RAM for data storage. The microprocessor 1302 can also be connected to a wireless communication interface 1318 for communicating with external devices such as a programmer, a home-based unit and/or a mobile unit (e.g., a cellular phone, personal computer, smart phone, tablet computer, and the like) or directly to the cloud or another communication network as facilitated by a cellular or other data communication network. The medical device can include a power supply circuit 1320. In some embodiments, the medical device can include an inductive energy receiver coil interface (not shown) communicatively coupled or attached thereto to facilitate transcutaneous recharging of the medical device.

The medical device can include one or more electric field sensing electrodes 1308 and one or more electric field sensor channel interfaces 1306 that can communicate with a port of microprocessor 1302. The medical device can also include one or more electric field generating circuits 1322, one or more electric field generating electrodes 1312, and one or more electric field generating channel interfaces 1310 that can communicate with a port of microprocessor 1302. The medical device can also include one or more temperature sensors 1316 and one or more temperature sensor channel interfaces 1314 that can communicate with a port of microprocessor 1302. The channel interfaces 1306, 1310, and 1314 can include various components such as analog-to-digital converters for digitizing signal inputs, sensing amplifiers, registers which can be written to by the control circuitry in order to adjust the gain and threshold values for the sensing amplifiers, source drivers, modulators, demodulators, multiplexers, and the like.

In some embodiments, one or more physiological sensors can also be included herein. In some embodiments, the physiological sensors can include sensors that monitor temperature, blood flow, blood pressure, pulse rate, and the like. In some embodiments, the respiration sensors can include sensors that monitor respiration rate, respiration peak amplitude, and the like. In some embodiments, the chemical sensors can measure the quantity of an analyte present in a treatment area about the sensor, including but not limited to analytes such as of blood urea nitrogen, creatinine, fibrin, fibrinogen, immunoglobulins, deoxyribonucleic acids, ribonucleic acids, potassium, sodium, chloride, calcium, magnesium, lithium, hydronium, hydrogen phosphate, bicarbonate, and the like. However, many other analytes are also contemplated herein. Exemplary chemical/analyte sensors are disclosed in commonly owned U.S. Pat. No. 7,809,441 to Kane et al., and which is hereby incorporated by reference in its entirety.

In some embodiments, other sensors can also be included herein such as one or more accelerometers or other types of motion sensors. In various embodiments, accelerometer or motion sensor data can be used to determine aspects of patient status and/or behavior such as their posture (lying down, sitting, standing, etc.), their current physical activity level (e.g., sedentary, walking, exercising, etc.), and the like. Such data can be used as described elsewhere herein with respect to activity-based therapy modulation.

Although the temperature sensors 1316 are shown as part of a medical device in FIG. 13, it is realized that in some embodiments one or more of the sensors could be physically separate from the medical device. In various embodiments, one or more of the can be within another implanted medical device communicatively coupled to a medical device via wireless communication interface 1318. In yet other embodiments, one or more of the sensors can be external to the body and coupled to a medical device via wireless communication interface 1318.

### Electric Field Therapy Parameters

In some embodiments, medical devices herein can generate one or more electric fields at frequencies selected from a range of between 10 kHz to 1 MHz. In some embodiments, the one or more electric fields can be effective to prevent and/or disrupt cellular mitosis in a cell. In some embodiments, the one or more electric fields can be effective to prevent and/or disrupt cellular mitosis in a cell, but not cause tissue ablation. In some embodiments, the system can be configured to deliver an electric field at one or more frequencies selected from a range of within 300 kHz to 500 kHz. In some embodiments, the system can be configured to deliver an electric field at one or more frequencies selected from a range of within 100 kHz to 300 kHz. In some embodiments, the system can be configured to periodically deliver an electric field using one or more frequencies greater than 1 MHz.

A desired electric field strength can be achieved by delivering an electric current between two electrodes. The specific current and voltage at which the electric field is delivered can vary and can be adjusted to achieve the desired electric field strength at the site of the tissue to be treated. In some embodiments, the system can be configured to deliver an electric field using currents ranging from 1 mAmp to 1000 mAmp to the site of a cancerous tumor. In some embodiments, the system can be configured to deliver an electric field using currents ranging from 20 mAmp to 500 mAmp to the site of a cancerous tumor. In some embodiments, the system can be configured to deliver an electric field using currents ranging from 30 mAmp to 300 mAmp to the site of a cancerous tumor.

In some embodiments, the system can be configured to deliver an electric field using currents including 1 mAmp, 2 mAmp, 3 mAmp, 4 mAmp, 5 mAmp, 6 mAmp, 7 mAmp, 8 mAmp, 9 mAmp, 10 mAmp, 15 mAmp, 20 mAmp, 25 mAmp, 30 mAmp, 35 mAmp, 40 mAmp, 45 mAmp, 50 mAmp, 60 mAmp, 70 mAmp, 80 mAmp, 90 mAmp, 100 mAmp, 125 mAmp, 150 mAmp, 175 mAmp, 200 mAmp, 225 mAmp, 250 mAmp, 275 mAmp, 300 mAmp, 325 mAmp, 350 mAmp, 375 mAmp, 400 mAmp, 425 mAmp, 450 mAmp, 475 mAmp, 500 mAmp, 525 mAmp, 550 mAmp, 575 mAmp, 600 mAmp, 625 mAmp, 650 mAmp, 675 mAmp, 700 mAmp, 725 mAmp, 750 mAmp, 775 mAmp, 800 mAmp, 825 mAmp, 850 mAmp, 875 mAmp, 900 mAmp, 925 mAmp, 950 mAmp, 975 mAmp, or 1000 mAmp. It will be appreciated that the system can be configured to deliver an electric field at a current falling within a range, wherein any of the forgoing currents can serve as the lower or upper bound of the range, provided that the lower bound of the range is a value less than the upper bound of the range.

In some embodiments, the system can be configured to deliver an electric field using voltages ranging from 1 Vrms to 50 Vrms to the site of a cancerous tumor. In some embodiments, system can be configured to deliver an electric field using voltages ranging from 5 Vrms to 30 Vrms to the site of a cancerous tumor. In some embodiments, the system can be configured to deliver an electric field using voltages ranging from 10 Vrms to 20 Vrms to the site of a cancerous tumor.

In some embodiments, the system can be configured to deliver an electric field using one or more voltages including 1 Vrms, 2 Vrms, 3 Vrms, 4 Vrms, 5 Vrms, 6 Vrms, 7 Vrms, 8 Vrms, 9 Vrms, 10 Vrms, 15 Vrms, 20 Vrms, 25 Vrms, 30 Vrms, 35 Vrms, 40 Vrms, 45 Vrms, or 50 Vrms. It will be appreciated that the system can be configured to deliver an electric field at a voltage falling within a range, wherein any of the forgoing voltages can serve as the lower or upper bound of the range, provided that the lower bound of the range is a value less than the upper bound of the range.

In some embodiments, the system can be configured to deliver an electric field using one or more frequencies including 10 kHz, 20 kHz, 30 kHz, 40 kHz, 50 kHz, 60 kHz, 70 kHz, 80 kHz, 90 kHz, 100 kHz, 125 kHz, 150 kHz, 175 kHz, 200 kHz, 225 kHz, 250 kHz, 275 kHz, 300 kHz, 325 kHz, 350 kHz, 375 kHz, 400 kHz, 425 kHz, 450 kHz, 475 kHz, 500 kHz, 525 kHz, 550 kHz, 575 kHz, 600 kHz, 625 kHz, 650 kHz, 675 kHz, 700 kHz, 725 kHz, 750 kHz, 775 kHz, 800 kHz, 825 kHz, 850 kHz, 875 kHz, 900 kHz, 925 kHz, 950 kHz, 975 kHz, 1 MHz. It will be appreciated that the system can be configured to deliver an electric field using a frequency falling within a range, wherein any of the foregoing frequencies can serve as the upper or lower bound of the range, provided that the upper bound is greater than the lower bound.

In some embodiments, the system can be configured to generate one or more applied electric field strengths selected from a range of within 0.25 V/cm to 1000 V/cm. In some embodiments, the system can be configured to generate one or more applied electric field strengths of greater than 3 V/cm. In some embodiments, the system can be configured to generate one or more applied electric field strengths selected from a range of within 1 V/cm to 10 V/cm. In some embodiments, the system can be configured to generate one or more applied electric field strengths selected from a range of within 3 V/cm to 5 V/cm.

In other embodiments, the system can be configured to deliver one or more applied electric field strengths including 0.25 V/cm, 0.5 V/cm, 0.75 V/cm, 1.0 V/cm, 2.0 V/cm, 3.0 V/cm, 5.0 V/cm, 6.0 V/cm, 7.0 V/cm, 8.0 V/cm, 9.0 V/cm, 10.0 V/cm, 20.0 V/cm, 30.0 V/cm, 40.0 V/cm, 50.0 V/cm, 60.0 V/cm, 70.0 V/cm, 80.0 V/cm, 90.0 V/cm, 100.0 V/cm, 125.0 V/cm, 150.0 V/cm, 175.0 V/cm, 200.0 V/cm, 225.0 V/cm, 250.0 V/cm, 275.0 V/cm, 300.0 V/cm, 325.0 V/cm, 350.0 V/cm, 375.0 V/cm, 400.0 V/cm, 425.0 V/cm, 450.0 V/cm, 475.0 V/cm, 500.0 V/cm, 600.0 V/cm, 700.0 V/cm, 800.0 V/cm, 900.0 V/cm, 1000.0 V/cm. It will be appreciated that the system can generate an electric field having a field strength at a treatment site falling within a range, wherein any of the foregoing field strengths can serve as the upper or lower bound of the range, provided that the upper bound is greater than the lower bound.

In some embodiments, an electric field can be applied to the site of a cancerous tumor or tumor resection at a specific frequency or constant frequency range.

In some embodiments, the electric field can be modulated in response to a patient's measured reference or core body temperature and/or a set period of time elapsing. For example, if the patient's reference or core body temperature is higher than a threshold level, the therapy parameters can be modulated to reduce the heat output of the system inside the body. If the patient's reference or core body temperature is higher than a threshold level, the electric field strength can be decreased by 5%, 10%, 15%, 20%, 25%, 30%, 35%, 40%, 45%, 50%, 55%, 60%, 65%, 70%, 75%, 80%, 85%, 90%, 95%, or 100% (e.g., turned off) or can be decreased by an amount falling within a range between any of the foregoing. If the patient's reference or core body temperature is higher than a threshold level, the electric field strength can be decreased by from 5% to 100%, or between 5% and 95%. It will be appreciated that other parameters can also be modulated in order to reduce the amount of heat generated by the system including, for example, current, voltage, and/or frequency.

Alternatively, if the reference or core body temperature is lower than a threshold level, then to maximize exposure to therapeutic electrical fields the electric field strength can be increased. In some embodiments, the electric field strength can be increased by 5%, 10%, 15%, 20%, 25%, 50%, 75%, 100%, 200%, 300%, 500%, 1000% or more, or by an amount falling within a range between any of the foregoing. It will be appreciated that other parameters can also be modulated in order to increase the intensity of the electrical field therapy provided by the system including, for example, current, voltage, and/or frequency.

### Temperature Models

As referenced above, some types of temperature fluctuations may be unique to an individual. Specifically, the magnitude of temperature change in response to certain events can be unique to an individual. For example, some individuals may regularly experience a core or reference temperature drop during sleep of a first magnitude. In contrast, other individuals may regularly experience a core or reference temperature drop during sleep of a different magnitude. In addition, based on differences in the exact implantation sites of electrodes, some individuals may experience an increase in temperature at a tissue site of a first magnitude for a given intensity level of therapy whereas other individuals may experience an increase in temperature of a different magnitude. Understanding how a given individual will likely experience temperature fluctuations can be extremely valuable to characterize and/or provide to a clinician and/or to automatically adjust therapy intensity by the system itself. In some embodiments, temperature data of an individual can be collected and recorded over a learning phase and then processed by the system and/or computing resources in data communication therewith in order to characterize the individual's unique temperature fluctuation response to electrical stimulation therapy and/or events such as fever, exercise, sleep and the like and/or produce a model of the same. Such characterizations and/or the model can then be used by the system and/or provided to a clinician in order to plan/program therapy to be delivered to the patient by the system. For example, such characterizations and/or the model can then be used by the system and/or provided to a clinician and used to accurately predict temperatures of tissues resulting from different intensities of electrical stimulation therapy as administered at various times.

Systems herein can process temperature data in various ways in order to characterize and/or derive a model regarding the same. In some embodiments, a predictive model can be developed by applying machine learning techniques or other statistical techniques such as predictive modeling to a data set of temperature data of the individual. Such data processing as model generation can be performed by the implantable system itself and/or by a computing resource (such as in the cloud) receiving data from the implantable system. In some embodiments, characterizations and/or a model can be generated using decision trees, regression models (linear and logistic), neural networks, and the like. In some embodiments, a supervised machine learning approach can be used to generate a regression model that provides a specific prediction of temperature change for an individual or a class of individuals based on an event or circumstance such as therapy provided a specific level of intensity, exercise, fever, or the like. In some embodiments, ensemble approaches can be used to characterize and/or build a temperature data model. Other techniques can also be applied herein.

Predictions of temperatures herein can apply to any relevant temperatures including, but not limited to, core temperatures, reference temperatures, temperatures at the site of electrodes, temperatures at various points within a zone of treatment and at various distances from the site of electrodes, and the like.

Beyond providing predictions of temperatures reached, in some embodiments the system can use the model to generate a specific recommendation of therapy intensity (therapy parameter values resulting in a therapy intensity) that can be used while not exceeding a threshold value of temperature (heat) exposure as a point-in-time absolute value and/or as an aggregate accumulated exposure value. In some embodiments, the recommendation can be presented to a system user such as a clinician for implementation. In some embodiments, the system can automatically implement such recommended values in order to keep temperature exposure below a threshold value.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

As used herein, the recitation of numerical ranges by endpoints shall include all numbers subsumed within that range (e.g., 2 to 8 includes 2.1, 2.8, 5.3, 7, etc.).

The headings used herein are provided for consistency with suggestions under 37 CFR 1.77 or otherwise to provide organizational cues. These headings shall not be viewed to limit or characterize the invention(s) set out in any claims that may issue from this disclosure. As an example, although the headings refer to a “Field,” such claims should not be limited by the language chosen under this heading to describe the so-called technical field. Further, a description of a technology in the “Background” is not an admission that technology is prior art to any invention(s) in this disclosure. Neither is the “Summary” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

1. An implantable system for cancer treatment comprising:
a therapy output circuit, wherein the therapy output circuit is configured to generate an electrical output for one or more electrodes to create one or more electric fields;
control circuitry, wherein the control circuitry causes the therapy output circuit to generate the one or more electric fields at frequencies selected from a range of between 10 kHz to 1 MHz within a bodily tissue, wherein the one or more electric fields are effective to prevent and/or disrupt cellular mitosis in a cell; and
a therapy zone temperature sensor;
wherein the implantable system is configured to measure temperature and/or record temperature data of a patient over time; and
the temperature data comprising tissue temperatures and time stamps of the same.

2. The implantable system of claim 1, wherein the control circuitry is configured to process the temperature data and modulate a therapy parameter based on the temperature data.

3. The implantable system of claim 2, wherein the implantable system is configured to use activity state data of the one or more electrodes and/or operational history of the one or more electrodes when processing temperature data from the therapy zone temperature sensor.

4. The implantable system of claim 2, the therapy parameter comprising at least one selected from the group consisting of a field strength, a duty cycle, and a therapy vector.

5. The implantable system of claim 1, wherein the control circuitry is configured to process the temperature data and modulate a therapy parameter based on the temperature data as part of a closed loop control system.

6. The implantable system of claim 5, the therapy parameter comprising a closed loop thermal setpoint value.

7. The implantable system of claim 1, wherein the implantable system is configured to index the temperature data based on one or more of direct or indirect measurement, tissue type, and electrode proximity.

8. The implantable system of claim 1, wherein the implantable system is configured to track the amount of time that tissue is exposed to temperatures above a threshold level.

9. The implantable system of claim 8, wherein the implantable system is configured to modulate a therapy parameter to limit the amount of time that the tissue is exposed to temperatures above a threshold level.

10. The implantable system of claim 8, wherein the implantable system is configured to reduce a therapy parameter value to limit the amount of time that the tissue is exposed to temperatures above a threshold level over a specific time period and then later increase the therapy parameter value.

11. The implantable system of claim 1, wherein the implantable system is configured to increase an intensity of the therapy at times when a core body temperature or a reference body temperature is lower than a threshold temperature.

12. The implantable system of claim 1, wherein the implantable system is configured to increase an intensity of a therapy parameter at times when the patient is asleep.

13. The implantable system of claim 1, wherein the implantable system is configured to decrease an intensity of a therapy parameter at times when the patient has an elevated core body temperature or reference body temperature.

14. The implantable system of claim 1, wherein the implantable system is configured to decrease an intensity of a therapy parameter at times when the patient has a fever.

15. The implantable system of claim 1, wherein the therapy zone temperature sensor is configured to measure or estimate a core body temperature or a reference body temperature when therapy is turned off or paused.

16. The implantable system of claim 1, further comprising:
a stimulation lead, the stimulation lead comprising an electrode; and
wherein the therapy zone temperature sensor is configured to measure a temperature at a site offset from a surface of the electrode.

17. The implantable system of claim 16, wherein the implantable system compensates for the offset in measuring or estimating tissue temperatures.

18. The implantable system of claim 1, further comprising an accelerometer;
wherein the implantable system is configured to estimate physical activity based on a signal from the accelerometer and modulate a therapy parameter based on the same.

19. The implantable system of claim 18, wherein the implantable system is configured to modulate the therapy parameter based on estimated physical activity in advance of measured temperature changes.

20. An implantable system for cancer treatment comprising:
a housing;
a therapy output circuit, wherein the therapy output circuit is configured to generate one or more electric fields; and
control circuitry;
wherein the control circuitry causes the therapy output circuit to generate the one or more electric fields at frequencies selected from a range of between 10 kHz to 1 MHz within a bodily tissue, wherein the one or more electric fields are effective to prevent and/or disrupt cellular mitosis in a cell;
wherein the control circuitry and the therapy output circuit are disposed within the housing;
a first temperature sensor, wherein the first temperature sensor is configured to measure a temperature of tissue at a site of therapy;
a stimulation lead;
stimulation electrodes;
wherein the stimulation electrodes are disposed on the stimulation lead;
wherein the stimulation electrodes are in electrical communication with the therapy output circuit; and
a second temperature sensor, wherein the second temperature sensor is configured to measure a core temperature or reference temperature of a patient.
